(19) United States
(12) Patent Application Publication
(10) Pub. No.: US 2025/0242103 A1
(43) Pub. Date: Jul. 31, 2025

(54) NASAL IRRIGATION DEVICE

(71) Applicant: The Procter & Gamble Company, Cincinnati, OH (US)

(72) Inventors: Kelly Lee SCHMEICHEL, Cincinnati, OH (US); Juan Jose BONILLA, Cincinnati, OH (US); Constanza Paz SANZ GONZALEZ, Costa Del Este (PA); Luis PERAZA, New Albany, OH (US); Drayton Jay SANTAVICCA, Newark, OH (US)

(21) Appl. No.: 19/039,458

(22) Filed: Jan. 28, 2025

Related U.S. Application Data
(60) Provisional application No. 63/627,120, filed on Jan. 31, 2024.

(51) Int. Cl. A61M 3/02

(52) U.S. Cl. CPC A61M 3/0279; A61M 2205/3334; A61M 2205/586; A61M 2210/0618

(57) ABSTRACT

A nasal irrigation device that includes a receptacle and a top that is selectively connectable to the receptacle, where the top includes a nozzle that is selectively rotatable to the receptacle, the nozzle including a finger placement area and a projection circumferentially located away from the finger placement area, the finger placement area and the projection being configured to facilitate rotation of the nozzle. Related processes of using and making such devices.

### FIELD OF THE INVENTION

The present disclosure relates to nasal irrigation devices with rotatable nozzles, related parts thereof, and processes related thereto.

### BACKGROUND OF THE INVENTION

Anyone with a nose knows that nasal passages can become irritated or congested for a variety of reasons, such as pollution, allergens, cold temperatures, dry climates, and/or a build-up of mucus due to infection. Nasal irrigation, in which a liquid such as saline solution is provided to the nasosinal cavities, can provide relief in such circumstances. For example, the nasal irrigation process can be helpful in flushing or washing impurities from the nasosinal cavities and/or hydrating the mucus membranes thereof, thereby improving the comfort and/or health of the user.

Although various devices are known to be useful for carrying out such nasal irrigation processes, known devices have various shortcomings. For example, a device commonly known as a “Neti pot,” which is shaped like a tea pot or an “Aladdin's lamp,” allows a liquid to flow into the nasosinal cavities by gravity; however, the gravity-based dispensing mechanism requires lifting and tilting the device above one's shoulders, which can be uncomfortable for some users. Some devices have an “always open” nozzle that does not allow for convenient mixing of salt and water in the receptacle portion of the device, e.g., through shaking the device. Other devices have “push-pull” nozzles that are either fully closed or fully open, meaning that a user is limited in his/her ability to control the flow rate of the liquid. Still other devices have nozzles that rotate to open, but such nozzles can be difficult to grip, particularly when wetted during use, e.g., with saline solution or mucus; additionally, a user may be confused as to which part of the device must be rotated.

In view of the foregoing, there is a need for improved nasal irrigation devices, parts thereof, and related process that solve for one or more of these challenges.

### SUMMARY OF THE INVENTION

The present disclosure relates to nasal irrigation devices with rotatable nozzles, related parts, and related processes of making such devices and/or parts.

For example, the present disclosure relates to a nasal irrigation device that includes: a receptacle, the receptacle comprising a body and an opening; and a top that is selectively connectable to the receptacle, the top including a nozzle that is selectively rotatable relative to the receptacle, the nozzle having a dispensing orifice located at a tip of the nozzle, the nozzle further having a finger placement area and a projection, where the finger placement area includes a centroid, where the projection is circumferentially located on the nozzle between 100° and 260°, preferably between 135° and 225°, preferably about 180°, from the centroid of the finger placement area, and where the finger placement area and the projection are configured to facilitate rotation of the nozzle relative to the receptacle.

The present disclosure also relates to a nasal irrigation device that includes a receptacle and a top having a nozzle that is rotatable relative to the receptacle, the nozzle including a dispensing orifice located at a tip of the nozzle, the nozzle further including a finger placement area, where the tip of the nozzle tilts away from a longitudinal axis and towards the finger placement area.

The present disclosure also relates to a nasal irrigation device that includes a receptacle and a top, the top having a nozzle and an intermediate part located between the nozzle and the receptacle, where the nozzle is rotatable relative to the intermediate part, where the nozzle is characterized by a first color and the intermediate part is characterized by a second color that is different from the first color.

The present disclosure also relates to a top for a nasal irrigation device, where the top includes a nozzle connected to an intermediate part, wherein the nozzle is selectively rotatable relative to the intermediate part. The nozzle may include a finger placement area and a projection, where the finger placement area includes a centroid, where the projection is circumferentially located on the nozzle between 100° and 260°, preferably between 135° and 225°, preferably about 180°, from the centroid of the finger placement area, and where the finger placement area and the projection are configured to facilitate rotation of the nozzle relative to the intermediate part; and the intermediate part being configured to be selectively connectable to a receptacle of a nasal irrigation device. The nozzle may include a finger placement area, where the tip of the nozzle tilts away from a longitudinal axis and towards the finger placement area. The nozzle may be characterized by a first color and the intermediate part may be characterized by a second color that is different from the first color.

The present disclosure also relates to a nasal irrigation kit that includes a nasal irrigation device according to the present disclosure, and a package that includes a salt.

The present disclosure also relates to a process of using a nasal irrigation device according to the present disclosure, where the process includes the steps of: providing a salt solution to the interior volume of the receptacle of the nasal irrigation device, preferably where the salt solution is prepared by providing a salt and water to the interior volume of the receptacle and mixing; attaching the top to the receptacle; inserting the tip of the nozzle into a human nostril; and dispensing at least a portion of the salt solution through the nozzle to the human nostril.

### BRIEF DESCRIPTION OF THE DRAWINGS

The figures herein are illustrative in nature and are not intended to be limiting.

FIG. 1 shows a nasal irrigation device according to the present disclosure.

FIG. 2 shows an exploded front view of a nasal irrigation device according to the present disclosure, including an optional tube.

FIG. 3A and FIG. 3B each show illustrative, alternative receptacle shapes for the devices according to the present disclosure.

FIG. 4 shows a front view of a top of a nasal irrigation device according to the present disclosure.

FIG. 5 shows a front view of an illustrative, alternative top.

FIG. 6 shows a rear view of a top of a nasal irrigation device according to the present disclosure.

FIG. 7 shows a side view of a top of a nasal irrigation device according to the present disclosure.

FIG. 8 shows a top perspective view of an intermediate part of a top according to the present disclosure.

FIG. 9 shows an exploded, side cross-sectional view of a top, as viewed at line W-W of FIG. 2.

FIG. 10 shows a top view of a top according to the present disclosure.

FIG. 11 shows a schematic cross-sectional view of a top, for example as viewed at line X-X of FIG. 10, where the nozzle is in a first, or closed, position.

FIG. 12 shows a schematic cross-sectional view of a top, where the nozzle is in a second, or open, position.

FIG. 13 shows a package suitable for containing salt.

### DETAILED DESCRIPTION OF THE INVENTION

The present disclosure relates to nasal irrigation devices, parts related thereto, and related processes. The nasal irrigation devices (or simply “device” or “devices” as used herein) include a nozzle that is selectively rotatable from a closed position to an open position.

Without wishing to be bound by theory, it is believed that the nasal irrigation devices described herein are convenient to use due to one or more of the features described below. In particular, it is believed that one or more of the features described below facilitate convenient and intuitive rotation of the nozzle from a closed position to an open position, and vice versa.

For example, the nozzles of the present disclosure may include a defined finger placement area that facilitates convenient and intuitive nozzle rotation, which may be located below an angled tip of the nozzle. The nozzle may further include second finger placement area, for example in the form of a projection, that can be operated in concert with the (first) finger placement area, enabling convenient rotation of the nozzle. Additionally or alternatively, the nozzle may be of a color that visually contrasts with the color of another portion of the device, such as an intermediate part or transition piece that is located between the receptacle of the device and the nozzle, thereby providing a signal to the user of which part should be rotated relative to another.

The nasal irrigation devices of the present disclosure, parts thereof, and related processes are described in more detail below.

As used herein, the articles “a” and “an” when used in a claim, are understood to mean one or more of what is claimed or described. As used herein, the terms “include,” “includes,” and “including” are meant to be non-limiting. The compositions of the present disclosure can comprise, consist essentially of, or consist of, the components of the present disclosure.

The terms “substantially free of” or “substantially free from” may be used herein. This means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or less than 0.1%, or less than 0.01%, or even 0%, by weight of the composition.

Unless otherwise noted, all component or composition levels are in reference to the active portion of that component or composition, and are exclusive of impurities, for example, residual solvents or by-products, which may be present in commercially available sources of such components or compositions.

All temperatures herein are in degrees Celsius (C) unless otherwise indicated. Unless otherwise specified, all measurements herein are conducted at 20° C. and under the atmospheric pressure.

In all embodiments of the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Nasal Irrigation Device

The present disclosure relates to a nasal irrigation device (or simply “device,” as used herein). As mentioned above, such devices are useful for providing a liquid, typically a saline solution, to the nasosinal passages and membranes thereof in a washing or rinsing process. The liquid is typically provided via one nostril, and may substantially exit the other.

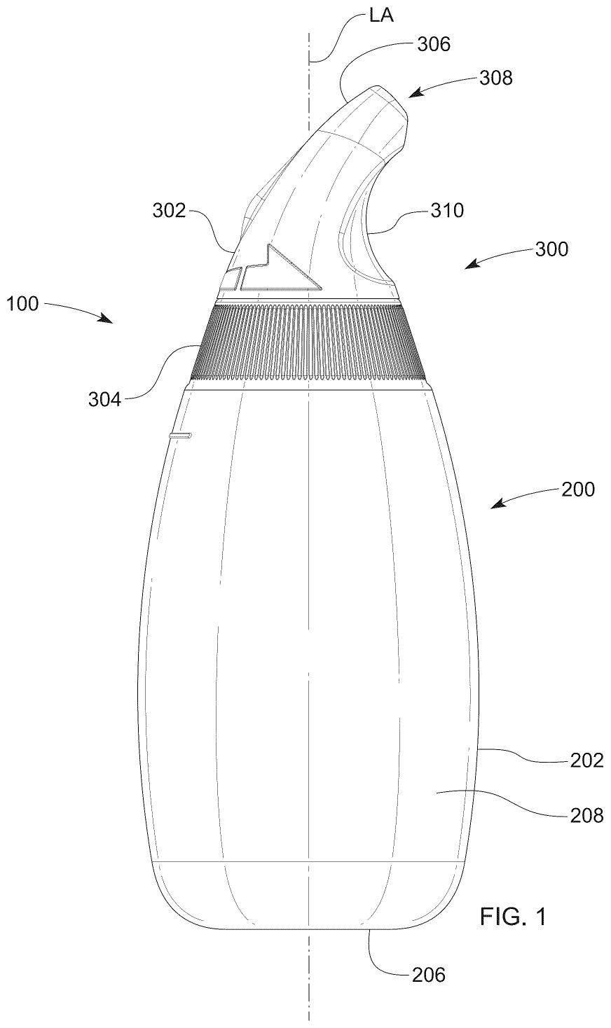

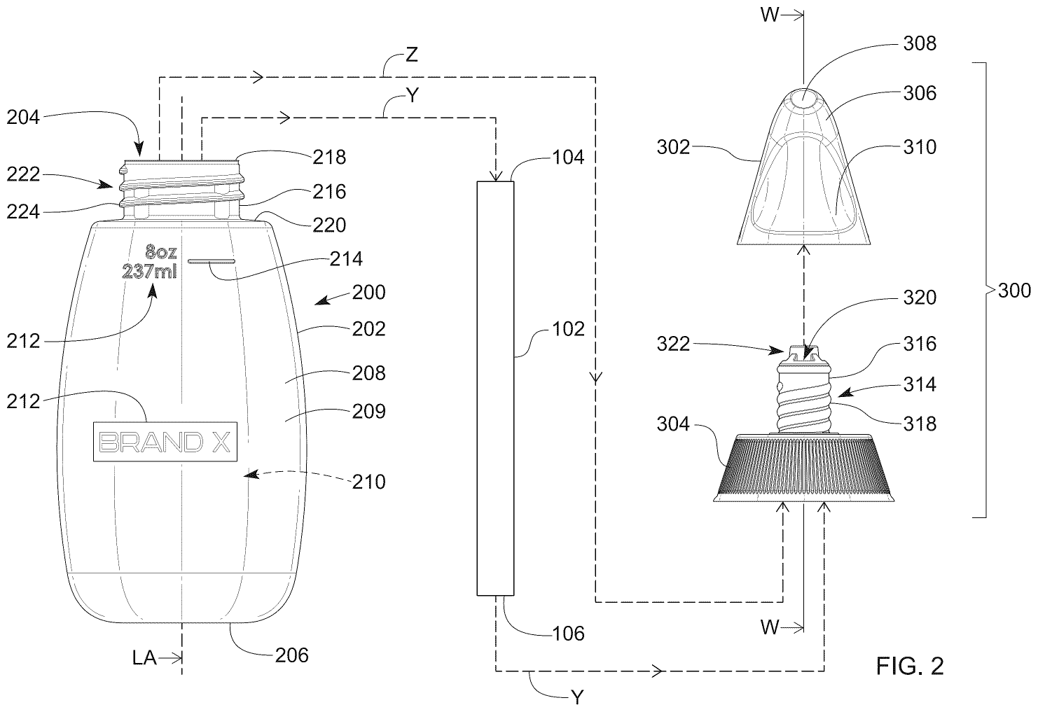

FIG. 1 shows an illustrative nasal irrigation device 100 according to the present disclosure, as viewed from the side. FIG. 2 shows an exploded view from the front of a nasal irrigation device 100 according to the present disclosure, including an optional tube 102. The device 100 comprises a receptacle 200 and a top 300. The receptacle 200 of the device 100 comprises a body 202 and an opening 204. The top 300 of the device 100 includes a nozzle 302 and an intermediate part 304. The various components and their features are described in more detail below.

The body 202 of the receptacle 200 comprises a base wall 206. The base wall 206 is typically configured to enable the receptacle 200 to stand upright on a flat surface. The base wall 206 may be substantially flat, or it may include one or more concave portions.

The body 202 further comprises at least one side wall 208 extending upwards from the base wall 206. The at least one side wall 208 may extend about a longitudinal axis LA that is substantially orthogonal to the base wall 206.

The at least one side wall 208 is preferably a flexible side wall. In other words, the at least one side wall 208, and preferably the body 202, is preferably made of a flexible material, such as plastic or silicone. A flexible side wall facilitates dispensing a fluid from the receptacle 200 via a squeezing process. Thus, when the optional tube 102 is in place, the nasal irrigation device 100 can be used in an upright position (e.g., where the top 300 is above the base wall 206). Further, when the nasal irrigation device 100 is used in an inverted position (e.g., where the base wall 206 is raised above the top 300), squeezing the at least one side wall 208 can supplement the pull of gravity, thereby giving the user more control over the flow rate.

The at least one side wall 208, preferably the body 202, more preferably the receptacle 200, may comprise a flexible material, preferably a flexible thermoplastic material, such as low density polyethylene (LDPE), high density polyethylene (HDPE), or polypropylene (PP), preferably LDPE. The receptacle 200 may be formed as a unitary piece, such as through injection molding, blow molding, or any other suitable process, preferably blow molding.

The at least one side wall 208, and preferably the body 202, is preferably at least partially translucent or transparent. Translucent or transparent slide walls 208 facilitate a user being able to see the amount of fluid that is present in the receptacle 200. The at least one side wall 208, preferably the body 202, may be at least partially opaque.

The base wall 206 and the at least one side wall 208 cooperate to define an interior volume 210 of the receptacle 200 that is accessible through the opening 204. The interior volume 210 may be of any size suitable for the intended purpose of the device, such as from about 100 mL to about 1 L, preferably from about 150 mL to about 500 mL, more preferably from about 200 mL to about 400 mL.

The body 202 may comprise indicia 212. The indicia 212 may comprise text, numerical values, shapes, symbols, or graphics. The indicia 212 may convey branding or marketing information (such as a logo or brand name), volumetric information, safety information, and/or instructional information. The indicia 212 may include a fill indicator 214 (shown in FIG. 2 as a fill line) that corresponds to a predetermined volume of fluid (e.g., water or saline solution). The indicia 212 may be printed on the body 202, preferably on an outer surface 209 of the at least one side wall 208. Additionally or alternatively, the indicia 212 may be molded on the body 202.

As shown in FIGS. 1 and 2, the body 202 may be generally cylindrical about the longitudinal axis LA. The at least one side wall 208 may be relatively convex.

The receptacle 200 may include a neck 216 extending away from the body 202. An upper edge 218 of the neck 216 may define the opening 204. The neck 216 may define a channel that connects the interior volume 210 of the body 202 with the opening 204. The longitudinal axis LA may pass through a center of the opening 204 and/or the neck 216. The upper edge 218 of the neck 204 may define a plane that is substantially parallel to the base wall 206 (or at least parallel to the flat surface upon which the base wall 206 rests to enable the receptacle to stand upright). The body 202 may include a shoulder 220 that connects the at least one side wall 208 with the neck 216.

The receptacle 200 includes a first connecting feature 222 that facilitates selectively removeable connection with the top 300. In the device of FIG. 2, the first connecting feature 222 is provided as one or more threads 224 on the neck 216 that facilitate a threadable connection with a top 300 according to the present disclosure.

The device 100 comprises a top 300. The top 300 is selectively connectable with the receptacle 200 in a manner that covers the opening 204 when the top 300 is connected to the receptacle 200.

The top 300 may comprise a second connecting feature 364 that facilitates selectively removeable connection with the receptacle 200. The second connecting feature 364 may be provided as one or more threads 366 that facilitate a threadable connection with the receptacle 300, for example with threads 224 on the neck 216 of the receptacle 200. Exemplary threads 366 of the top 300 (specifically, on the intermediate part 304) may be seen in the cross-sectional view of FIG. 9.

The top 300 of the device 100 comprises a nozzle 302. The nozzle 300 is selectively rotatable relative to the receptacle 200. The nozzle 300 may have a first position, which may correspond to a closed position in which liquid cannot flow out of the nozzle, and a second position, which may correspond to an open position in which liquid is able to flow out of the nozzle. The nozzle 300 may be selectively rotatable around a rotation axis from the first position to the second position and back. (See further description below with regard to FIGS. 9-12.) The rotation axis may substantially align with the longitudinal axis LA of the receptacle 200; in other embodiments, it may not. In FIG. 2, the rotation axis substantially aligns with line W-W.

The nozzle 302 includes a tip 306 and a dispensing orifice 308 located at the tip 306. When the top 300 is connected to the receptacle 200, the tip 306 is distally located relative to the receptacle 200.

The tip 306 is sized and dimensioned to fit into a user's nostril and preferably is capable of forming a relatively tight seal with the nostril so that liquid dispensed from the device 100 is restricted from exiting the same nostril when the device is so placed. In order to accommodate differently sized nostril openings of different users, the tip 306 of the nozzle 302 may have a relatively small cross-sectional area near the dispensing orifice 308 and a cross-sectional area that becomes gradually larger farther away from the dispensing orifice 308. Thus, the tip 306 of the nozzle 302 may be generally cone-shaped.

The nozzle 302 comprises a finger placement area 310. The nozzle 302 may preferably comprise a projection 312. The finger placement area 310 and the projection 312 are discussed in more detail below.

The top 300 may further comprise an intermediate part 304. When the top 300 is connected to the receptacle 200, the intermediate part 304 is located between the nozzle 302 and the receptacle 200. Put another way, when the top 300 is connected to the receptacle 200, the intermediate part 304 is proximally located relative to the receptacle 200.

The intermediate part 304 is connectable, optionally selectively connectable, to the nozzle 302. Once connected, it may be preferred for the nozzle 302 and intermediate part 304 to remain connected to facilitate a tight and relatively permanent connection. That being said, even when connected to the intermediate part 304, the nozzle 302 is typically selectively rotatable relative to the intermediate part 304, e.g., from a first/closed position to a second/open position. When the top 300 is connected to the receptacle 200, the receptacle 200 and the intermediate part 304 may cooperate as a single piece, and the nozzle 302 may be rotatable relative to the single piece formed therefrom.

The intermediate part 304 may comprise a nozzle-connecting feature 314 that facilitates connection with the nozzle 302. If the nozzle 302 is desired to rotate relative to the intermediate part 304, the nozzle-connecting feature 314 should be configured to allow such rotation. As shown in FIG. 2, the intermediate part 304 may comprise a post 316. The post 316 may comprise the nozzle-connecting feature 314. The nozzle-connecting feature 314 may comprise threads 318.

The intermediate part 304 comprises at least one opening 320 through which liquid can pass into the nozzle 302 and eventually be dispensed through the dispensing orifice 308. The at least one opening 320 may be located at a distal end 322 of the post 316.

The top 300 or parts thereof may be made of any suitable material, preferably a thermoplastic material. Suitable thermoplastic materials may include polypropylene (PP) or high density polyethylene (HDPE), preferably polypropylene (PP). The top 300 may be made according to any suitable process, such as injection molding or blow molding, preferably injection molding. The nozzle 302 and intermediate part 304, if present, may be made with the same type material or with different types of materials, preferably the same type materials (although the colors may differ, as described in more detail below). The nozzle 302 and intermediate part 304 may be made according to the same type process or according to different types of processes.

FIG. 2 further shows a tube 102. The tube 102 may be optional in devices 100 according to the present disclosure. The tube 102, when present, is selectively connectable to the top 300. When so connected, for example at a first end 104 of the tube 102, the tube 102 can extend into the interior volume 210 of the receptacle 200 when the top 300 is connected to the receptacle 200. Liquid contained in the interior volume 210 can enter a second end 106 of the tube 102 and exit to the top 300, for example when the device 100 is squeezed. Thus, the tube 102 is particularly useful when the device 100 is used in an upright position; in such cases, the device 100 may be assembled according to path Y shown in FIG. 2. If it is desirable for the device 100 to be used in an inverted position, then the tube 102 may be removed prior to use; in such cases, the device 100 may be assembled according to path Z shown in FIG. 2. Given that the device 100 could be exclusively used in an inverted position, the tube 102 is optional but is preferably available to facilitate more flexible usage of the device 100.

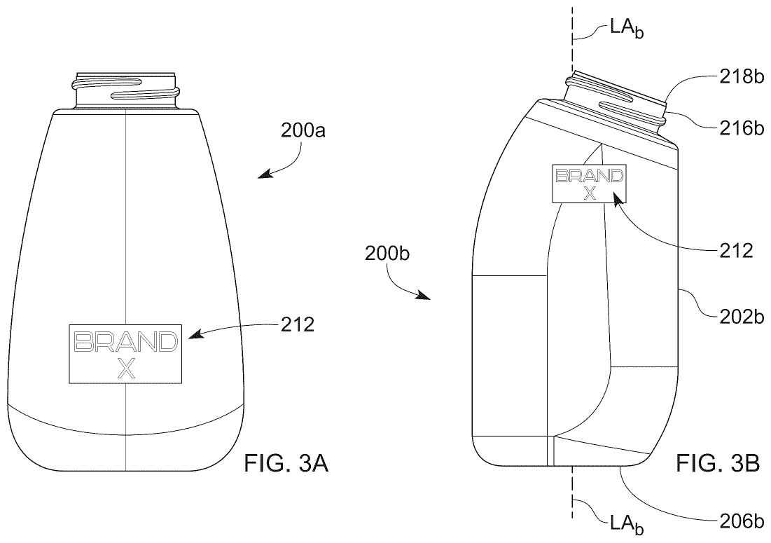

FIGS. 3A and 3B show illustrative, alternative shapes for receptacles 200a, 200b according to the present disclosure. Compared to the somewhat-cylindrical or ovoid-shaped receptacle 200 of FIGS. 1 and 2, the receptacle 200a of FIG. 3A is characterized by a profile that is a bit more triangular or pear-shaped, which may result in a relatively lower center of gravity and improved stability relative to tipping over. The receptacle 200b of FIG. 3B includes a neck 216b that tilts away from the longitudinal axis LA_{b} of the body 202b such that the plane defined by the upper edge 218b of the neck 216b is not parallel to the base wall 206b. This configuration may facilitate more convenient dispensing for some users, as the receptacle 200b may not need to be tilted as much as the receptacle 200 of FIG. 2.

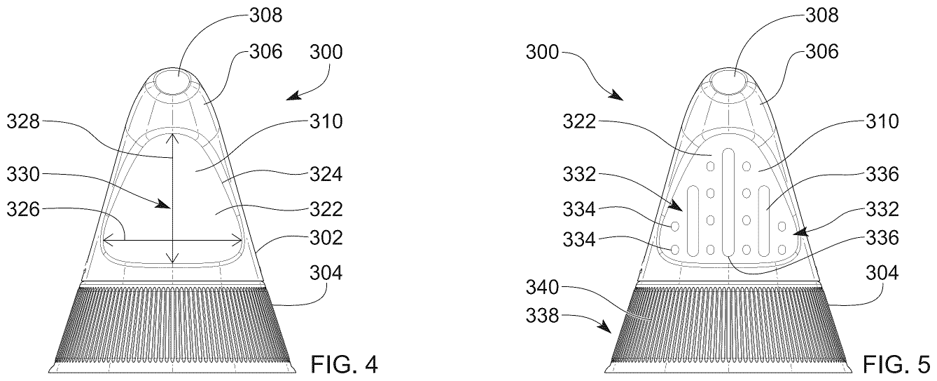

FIGS. 4-7 show tops 300 according to the present disclosure. As described above, the tops 300 include a nozzle 302 and an intermediate part 304.

As shown in FIG. 4, the nozzle 302 includes a finger placement area 310. The finger placement area 310 is sized and dimensioned to receive a user's fingertip on a surface 322 of the finger placement area 310, particularly when rotating the nozzle 302. The finger placement area 310 may comprise an outer edge 322 that defines the shape of the finger placement area 302. The outer edge 322 is preferably a continuous edge, although in some embodiments it may be discontinuous and/or fade into the nozzle 302 at certain points. The finger placement area 310 may be characterized by a shape. The shape may be defined, at least in part, by some or all of the outer edge 322. The shape of the finger placement area 310 may be a polygon, or the shape may not be a polygon. It may be preferred that the shape does not have any hard corners or vertices; instead, it may be preferred that the shape comprises one or more curves, which may feel more pleasant to a user's fingertips.

The outer edge 322 may define a perimeter of the finger placement area 310. The finger placement area 310 may be characterized by a surface area, which may be bounded by the perimeter and/or the outer edge 322. The surface area of the finger placement area 310 may be from about 50 mm² to about 2000 mm², preferably from about 100 mm² to about 1000 mm², more preferably from about 200 mm² to about 600 mm².

The finger placement area 310 may be characterized by a maximum width 326, measured along the surface 322 of the finger placement area 310 in the circumferential direction around the longitudinal axis. The maximum width 326 may be from about 10 mm to about 40 mm, preferably from about 20 mm to about 30 mm. The finger placement area 310 may be characterized by a maximum height 328, measured along the surface 322 of the finger placement area 310 in the longitudinal direction. The maximum height 328 may be from about 10 mm to about 50 mm, preferably from about 20 mm to about 40 mm. The finger placement area 310 may comprise a centroid 330.

The surface 322 of the finger placement area 310 of the nozzle 302 of FIG. 4 is relatively smooth, which may provide pleasant haptic interactions to a user. The surface 322 may be partially or completely smooth (e.g., a portion or the entirety of the surface is smooth).

The surface 322 of the finger placement area 310 may comprise surface irregularities 332. Surface irregularities 332 may be useful for improving friction and grip when rotating the nozzle 302, which may be especially convenient when the nozzle is wet from usage. Preferably, the surface irregularities 322 may comprise ridges, bumps, depressions, or combinations thereof. In FIG. 5, the surface 322 of the finger placement area 310 of the nozzle 302 includes illustrative surface irregularities 322 in the form of bumps 334 and ridges 336. However, it is understood that any suitable type and configuration of surface irregularities 332 may be employed by one of ordinary skill.

The intermediate part 304 may include an exterior surface 338. The exterior surface 338 may be substantially circumferentially located around the longitudinal axis LA. The exterior surface 338 may provide a gripping surface that a user may grip when connecting the top 300 to the receptacle 200, for example by rotating the top 300 relative to the receptacle 200. Additionally or alternatively, the exterior surface 338 of the intermediate part 304 may be gripped to hold the intermediate part 304 (and likely the receptacle 200) substantially stationary 304 while the nozzle 302 is rotated. The exterior surface 338 may include irregularities 340 that facilitate better grip. The irregularities 340 shown, e.g., in FIG. 5 are in the form of ridges that substantially extend longitudinally, but any suitable type or configuration of irregularities 340 (including ridges, bumps, or depressions) may be employed by one of ordinary skill.

The nozzle 302 and the intermediate part 304 may be visually contrasting, preferably with regard to visually contrasting coloration. It is believed that coloration differences between the nozzle 302 and the intermediate part 304 contribute to more instinctive and convenient usage. For example, a difference in color may signal to the user that the components (nozzle 302 and intermediate part 304) are separate and distinct, thereby suggesting that one may be rotated relative to the other. Thus, for tops 300 according to the present disclosure, the nozzle 302 may be characterized by a first color, and the intermediate part is preferably characterized by a second color that is different and/or visually distinct from the first color. It may be desirable that the first and second colors are different shades of the same general color (for example, lighter and darker shades of blue) for aesthetic or branding reasons. It may be desirable that the first and second colors are not different shades of the same general color but are instead different colors altogether (for example, red and blue), which may also be for aesthetics, branding reasons, or increased visual contrast between the components.

FIG. 6 shows a rear view of a top 300 according to the present disclosure. The nozzle 302 comprises a second finger placement area 311. The second finger placement area 311 is circumferentially located relative to the finger placement area 310 discussed above, which in such cases may be considered a first finger placement area. The second finger placement area 311 can provide a second location to conveniently grip and/or rotate the nozzle 302, independent of or preferably in concert with the (first) finger placement area 310.

The second finger placement area 311 may take any suitable form and may comprise one or more projections, depressions, or a combination thereof. The second finger placement area 311 preferably comprises a projection 312. A projection 312 may have any suitable form, such as one or more bumps, ridges, or combination thereof. Without wishing to be bound by theory, it is believed that coupling a finger placement area 310 that is concave in at least one direction (as shown, the longitudinal direction) with second finger placement area 311 in the form of a projection 312 facilitates a convenient and intuitive user experience.

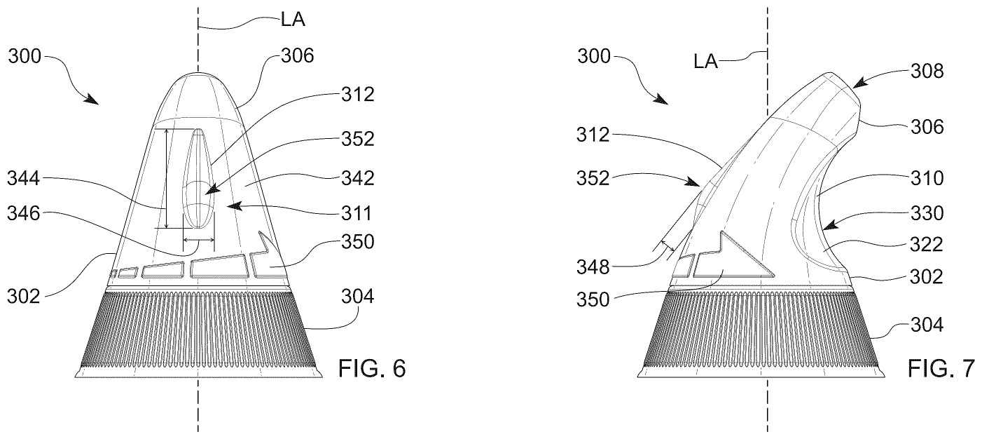

The nozzle 302 may comprise a second finger placement area 311 in the form of a projection 312 that extends outwardly away from an exterior surface 342 of the nozzle 302, as illustratively shown in FIGS. 6 and 7.

The projection 312 may be characterized by a projection length 344, a projection width 346, and/or a projection height 348. The projection length 344 is the maximum length of the projection 312, measured in a substantially longitudinal direction. The projection width 346 is the maximum width of the projection 312, measured in a substantially circumferential direction relative to the longitudinal axis. The projection height 348 is the maximum height of the projection 312, measured in a direction away from the exterior surface 342 of the nozzle 302.

The projection 312 may be in the form of a ridge, which can be characterized by a ratio of projection length 344 to projection width 346 that is greater than 1, preferably greater than 2. The projection 312 shown in FIG. 6 is in the form of a ridge. Preferably, a projection 312 in the form of a ridge is longitudinally oriented, e.g., extending towards the tip 306 and the base wall 206. Such an orientation is desirable, as it will provide a relatively large surface area for a user to push or pull against when rotating the nozzle 302. Put another way, it may be advantageous for the largest dimension (preferably the projection length 344) of the projection 312 to be oriented in a direction that is substantially orthogonal to the direction in which force must be applied to rotate the nozzle 302 in an opening or closing operation.

The second finger placement area 311, preferably a projection 312, is typically circumferentially located away from the finger placement area 310. The second finger placement area 311, preferably a projection 312, may be located at least about 90°, preferably at least about 100°, even more preferably at least about 135°, even more preferably about 180° in the shortest circumferential direction away from the finger placement area 310, preferably measured from the centroid 330 of the finger placement area 310 to a centroid 352 of the second finger placement area 311, preferably a centroid 352 of a projection. (It is understood that the centroid 330 of the finger placement area 310 and the centroid 352 of the second finger placement area 311/projection 312 may be longitudinally displaced from each other.) Without wishing to be bound by theory, it is believed that placing the second finger placement area 311, preferably a projection 312, away from the (first) finger placement area 310, preferably on an opposite side of the nozzle 302, will provide a user with the most convenient gripping surfaces when rotating the nozzle 302.

The top 300 may comprise indicia 350. The indicia 350 may be located on the nozzle 302, the intermediate part 304 (if present) or both. The indicia 350 may comprise instructional indicia, branding/marketing indicia (e.g., indicating a brand name), safety indicia, or combinations thereof. Instructional indicia may include, for example, directional indicia (e.g., indicating which direction to rotate the nozzle), flow indicia (e.g., indicating high, low, and/or zero flow rates), status indicia (e.g., indicating a closed position and/or an open position), or combinations thereof.

As shown in FIG. 7, the finger placement area 310 may be curved. The finger placement area 310 may be characterized by at least one of the following characteristics: (a) being substantially concave in a longitudinal direction; (b) being substantially convex in a circumferential direction; (c) both (a) and (b). Option (c) (being both substantially concave in a longitudinal direction and substantially convex in a circumferential direction) may be preferred for reasons of user comfort. Such a configuration may be described as “saddle-shaped.” Therefore, the finger placement area 310 may be saddle-shaped. For clarity, “saddle-shaped” as used in the present disclosure relates to the relative curvature of the surface 322 of the finger placement area 310 and does not necessarily anything about the perimeter, outer edge 324, or general (two-dimensional) shape of the finger placement area 310.

As also shown (although not exclusively) in FIG. 7, the tip 306 of the nozzle 302 may tilt away from the longitudinal axis LA. Such configurations can facilitate convenient usage of the present devices 100, for example comfortable nostril insertion or less mess during a nasal irrigation process (e.g., less fluid and/or drainage is directed to the device 100 and/or the user's hand).

Preferably, the tip 306 of the nozzle 302 tilts towards the finger placement area 310. In such configurations, the curve of the nozzle 302 can result in more accurate and/or secure finger placements as the user's finger is effectively guided to the finger placement area 310 by the overhanging tip 306. In such configurations, the tip 306 may also be used as a point of leverage to push or pull against during a rotation operation. In short, the described configuration can lead to easier, more comfortable, and/or more effective rotation of the nozzle 302.

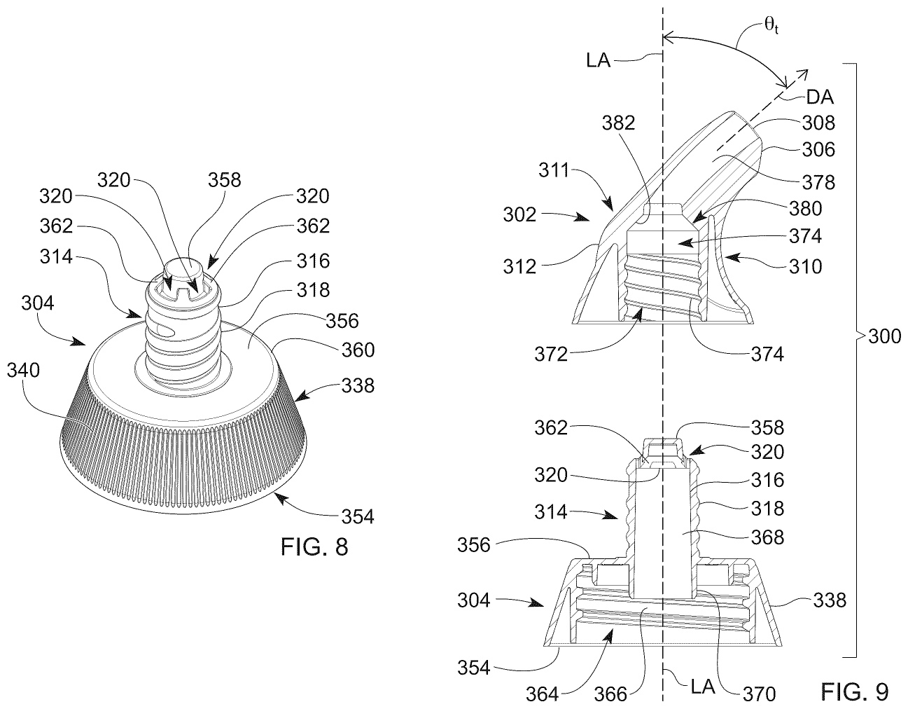

FIG. 8 shows a top perspective view of an intermediate part 304 according to the present disclosure. The intermediate part 304 includes a proximate side 354 that is oriented towards the receptacle 200 when connected to the receptacle200, and a distal side 356 that is oriented away from the receptacle 200. An exterior surface 338 extends between the proximate and distal sides 354, 356. The exterior surface 338 may include irregularities 340 that can facilitate convenient rotation. The exterior surface 338 may transition to the distal side 356 at a shoulder 360. A post 316 extends upwards from the distal side 356 of the intermediate part 304. The post 316 may terminate in a closed end 358. The closed end 358 may be held in place by one or more supports 362. The intermediate part 304 includes one or more openings 320 at the distal end of the intermediate part 304; the one or more openings 320 facilitate liquid traveling from the intermediate part 304 to the nozzle 302. The one or more openings may be near the closed end 358. The one or more openings 320 may be axially below (e.g., relatively more proximal to the proximate side than) the closed end 358. The one or more openings 320 may be framed, at least in part, by one or more supports 362. The intermediate part 304 may comprise two, preferably three, more preferably four openings 320. It is believed that the disclosed configuration of the openings 320 balances control of liquid flow with structural stability.

FIG. 9 shows a cross-section of an exploded side view of a top 300 according to the present disclosure, for example as taken and viewed along line W-W in FIG. 2. The top 300 comprises a nozzle 302 and an intermediate part 304.

As shown in the cross-section, the intermediate part 304 comprises a second connection feature 364, shown in the form of complementary threads 366, that can selectively connect with the first connection feature 22 of the receptacle 200. In addition to the one or more openings 320 (which are distal to the receptacle 200 when connected), the intermediate part 304 comprises a hollow channel 368 and an open end 370 (which is proximal to the receptacle 200 when connected). The open end 370, the hollow channel 368, and the one or more openings 320 cooperate to allow fluid communication from the receptacle 200 to the nozzle 302. The post 316 may comprise the hollow channel 368 and the open end 370. The post 316 may extend below the distal surface 356, and the tube 102, if present, may selectively connect to the open end 370.

As shown in the cross-section, the nozzle 302 comprises an intermediate-part-connecting feature 372, shown in the form of complementary threads 374, which can selectively connect to the nozzle-connecting feature 314 of the intermediate part 304, while still enabling rotation of the nozzle 302 relative to the intermediate part 304.

The nozzle 302 comprises a hollow interior portion 376. The hollow interior portion 376 may be configured to receive at least a portion of the intermediate part 304, such as the post 316 and/or the nozzle-connecting features 314.

The nozzle 302 further comprises a channel 378. At least a portion of the channel 378 is located in the tip 306 of the nozzle 302 and connects to the dispensing orifice 308. The channel 378 is configured to facilitate fluid communication through the nozzle 302. Thus, liquid that is dispensed from the device 100 can travel through the channel 378 and out of the dispensing orifice 308. The channel 378 may be connected to, or even be a part of, the hollow interior portion 376 of the nozzle 302.

As shown in FIG. 9, the tip 306 of the nozzle 302 tilts away from the longitudinal axis LA and towards the finger placement area 310. The tilted orientation results in the dispensing orifice 308 dispensing liquid along a dispensing axis DA that is not parallel to the longitudinal axis LA. The dispensing axis DA may be substantially orthogonal to a plane defined by the dispensing orifice 308 or a rim of the tip 306 that surrounds the dispensing orifice 308. The dispensing axis DA may parallel or even overlap with at least a portion of a central axis of the channel 378, depending on the configuration of the channel 378.

A tilt angle et of the tip 306 may be determined, which is the angle between by the longitudinal axis LA and the dispensing axis DA as shown in FIG. 9. The tilt angle O, of the tip 306 may be from about 1° to about 135°, preferably from about 10° to about 90°, more preferably from about 25° to about 65°, more preferably from about 35° to about 55°, even more preferably about 45°. Preferred tilt angles θₜ may provide an improved user experience.

The top 300 may comprise a flow-rate control feature 380. The flow-rate control feature 380 can control the flow rate of the liquid to be dispensed. The flow-rate control feature 380 can block the flow of liquid, for example, by blocking liquid from entering the channel 378 of the nozzle 302 and/or exiting from the one or more openings 320 of the intermediate part 304. The flow-rate control feature 380 can be manipulated by rotating the nozzle 302. For example, when the nozzle 380 is rotated to a first or closed position, the flow-rate control feature 380 may block or otherwise stop fluid communication through the top 300. When the nozzle 302 is rotated to a second or open position, the flow-rate control feature 380 no longer prevents fluid communication through the top 300, and the liquid may flow through and be dispensed.

As shown in FIG. 9, the flow-rate control feature 380 comprises an abutment surface 382 located in the nozzle 302. The abutment surface 382 is configured to block fluid communication through the nozzle 302 when the nozzle is in a first or closed position. In the top of FIG. 9, it does so by abutting the post 316 of the intermediate part 304 and blocking the one or more openings 320, for example by creating a seal in cooperation with the post 316, supports 362, and/or the closed end 358. When the nozzle 302 is rotated to a second or open position, the nozzle 302 moves distally up the post 316, and the abutment surface 382 no longer blocks or seals the one or more openings 320; thus, liquid can flow through the one or more openings 320 and into the channel 378 of the nozzle 302.

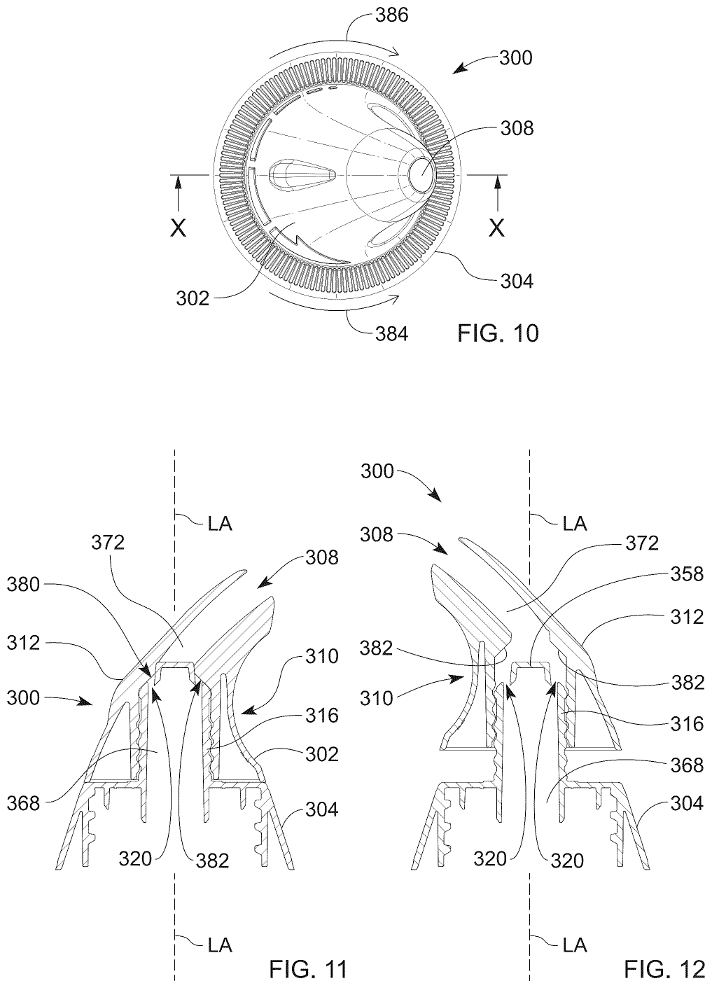

FIGS. 10, 11, and 12 substantially show an illustrative opening and closing mechanism of a top 300 according to the present disclosure. FIG. 10 shows a top view of a top 300 according to the present disclosure, including the nozzle 302 and the intermediate part 304. The nozzle 302 may be selectively rotated relative to the intermediate part 304 in a first direction 384 (e.g., an opening direction) from a first/closed position to a second/open position. The nozzle 302 may be selectively rotated relative to the intermediate part 304 in a second direction 386 (e.g., a closing direction) that is counter to the first direction 384 from the second/open position to the first/closed position. Rotation in the first and second directions 384, 386 may be around a rotation axis, which may correspond to the longitudinal axis LA.

FIG. 11 shows a schematic cross-sectional view of a top 300, for example as viewed at line X-X of FIG. 10. In FIG. 11, the nozzle 302 is in a first position, which corresponds to a closed position. The flow-rate control feature 380 of the nozzle 302 comprises an abutment surface 382. With the nozzle 302 in the first position (e.g., closed position), the abutment surface 382 blocks fluid communication through the top 300 by blocking or sealing the one or more openings 320 of the post 316 of the intermediate part 304. In FIG. 11, the abutment surface 382 creates a seal in cooperation with the post 316, including the closed end 358.

FIG. 12 shows a schematic cross-sectional view of the top 300, where the nozzle 302 is in a second position, which corresponds to an open position. The cross-section may be viewed substantially as shown in line X-X of FIG. 10, but here the nozzle 302 has been rotated, for example 180° or 540°, relative to the intermediate part 304 compared to the top 300 shown in the cross-section of FIG. 11. As a result of rotating the nozzle 302 in a first direction 384 (or opening direction), preferably facilitated by a user placing fingers on the finger placement area 310 and the projection 312, the nozzle 302 has moved upwards relative to (e.g., away from) the intermediate part 304 due to the threaded connection therebetween. This results in the flow-rate control feature 380 allowing fluid communication through the top 300. Specifically, the abutment surface 382 of the nozzle 302 is moved away from the post 316 and the closed end 358, thereby breaking the seal and allowing fluid communication from the hollow channel of the post 316, through the one or more openings 320 of the post 316, into the channel 372 of the nozzle 302, ultimately out of the dispensing orifice 308 and to the environment.

To close the top 300, e.g., for storage or mixing purposes, the nozzle 302 may be rotated in the second direction 386 (or closing direction) from the second (or open) position to the first (or closed) position so that fluid communication through the top 300 is blocked.

The nozzle 302 may be capable of being rotated to one or more intermediate positions that are in between the first/closed and second/open positions. In such cases, the second/open position is considered a fully open position, and the intermediate position(s) may be considered partially open position(s). When one or more intermediate positions are available, the flow rate may be partially restricted. For example, in the top 300 of FIG. 9, the abutment surface 382 may only partially block the one or more openings 320 when the nozzle 302 is in an intermediate position, and/or the flow-rate control feature 380 may allow constricted entrance to the channel 378. The possibility of one or more intermediate positions and/or partially restricted flow rates may be advantageous to the user because it provides the user with more control over the usage and flow rate of the device 100.

### Nasal Irrigator Top

The present disclosure further relates to a top 300 for a nasal irrigator device 100, as it is recognized that tops 300 according to the present disclosure may be manufactured and/or sold separately from a receptacle 100.

A top 300 for a nasal irrigator according to the present disclosure includes a nozzle 302. The top 300 may further include an intermediate part 304. Suitable tops 300 and components thereof are described in more detail above, but the disclosure will not be repeated for sake of brevity.

### Nasal Irrigation Kit

The present disclosure also relates to a kit useful for nasal irrigation processes (e.g., a nasal irrigation kit, or simply “kit” as used herein). The kit comprises a nasal irrigation device 100 according to the present disclosure, and a package 400; the package 400 preferably comprises salt. More than one of such packages 400 may be present. The device 100 and the one or more packages 400 of salt may be co-packaged in secondary packaging. The secondary packaging may be a box, which may be made of a paper-based material.

Suitable nasal irrigation devices 100 are described in more detail above, and the disclosure therein substantially applies to devices found in the presently described kits.

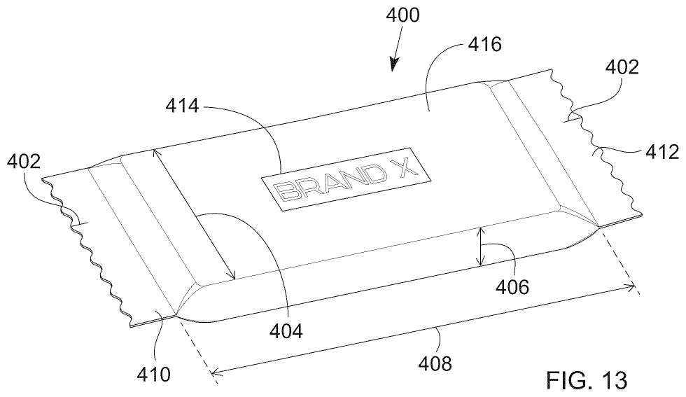

The kit further comprises a package 400 that contains a salt. The kit may comprise a plurality of such packages 400. For refill purposes, such packages 400 may also be sold separately from the nasal irrigation devices 100. FIG. 13 shows an exemplary package 13 that contains a salt.

The salt may comprise a compound selected from the group consisting of sodium chloride, magnesium chloride, potassium chloride, calcium chloride, sodium bicarbonate, and a combination thereof. The salt preferably comprises sodium chloride.

The package 400 typically comprises a predetermined amount of the salt. The predetermined amount of salt may be from about 0.1 g to about 5 g, preferably from about 1 g to about 4 g, more preferably from about 2 g to about 3 g, even more preferably about 2.13 g.

Preferably, the predetermined amount of salt in the package 400 is selected to provide a predetermined concentration in a salt solution of a predetermined volume. Preferably, the predetermined concentration corresponds to a (physiologically) isotonic solution (approx. 0.9% by wt.), a hypertonic solution (above 0.9% by wt.; for example, 2.5% by wt.), or a hypotonic solution (below 0.9% by wt.). Selection of the predetermined concentration and/or whether the resulting salt solution is isotonic, hypertonic, or hypotonic may depend on the needs or condition of the user and the desired treatment. One or more packages 400, for example two packages 400, may provide the predetermined amount of salt.

The predetermined amount of salt in the package 400 may be sufficient to provide a predetermined concentration in a salt solution that is made by mixing the predetermined amount of salt with water provided in an amount such that the resulting salt solution fills the interior volume 210 of the receptacle 200 to the fill line 214, which indicates a predetermined volume. For example, when the predetermined volume indicated on the receptacle 200 is approximately 240 mL (e.g., 237 mL or 8 oz), it may be desirable to provide approximately 2 g (e.g., 2.13 g) of salt and combine with sufficient water to meet the fill line 214 indicating the predetermined volume, thereby providing a resulting salt solution of approximately 0.9 wt % salt.

The package 400 may be of any suitable type, but packages400 in the form of sachets may be preferred. The package 400 shown in FIG. 13 is in the form of a sachet. Such packages 400 can be readily opened by a user, for example by tearing. The package 400 may comprise an accessibility feature 402 that facilitates convenient opening. For example, the accessibility feature 402 may comprise a line of weakness (such as a scored line or a perforation that can be easily torn), a partial tear or pre-formed notch in the package 400 (which effectively gives the user a “head start” when opening the package), or a combination thereof. In FIG. 13, the accessibility feature 402 is in the form of a partial tear. The accessibility feature 402 may be at or near a closed end 410, 412 of the package 400.

The package 400 may be made of a flexible material. Desirable materials include materials that can conveniently be sealed by the manufacturer and/or opened by the user (e.g., by tearing the material), preferably both. Suitable materials may include cellulosic-based material (e.g., a paper-based material), polyethylene (PE), foil, or a combination thereof. The package 400 may comprise a multi-layer laminate material with two or more layers. It may be preferred that the multilayer laminate material comprises an outer paper layer, which may facilitate convenient printing thereon and/or have a desirable environmental profile. The multilayer laminate material may comprise a foil layer, which may inhibit moisture intrusion that can have a negative impact on the salt contained in the package 400.

The package 400 may include a body 418 in which the salt is contained. The body 418 may be sized and configured so that once opened, the contents (e.g., salt) can be conveniently poured into the receptacle 200 of the nasal irrigator device 100. Thus, it may be preferred that the body 418 of the package 400 has at least a first dimension 404 (such as a width) preferably at least a first dimension 404 (e.g., width) and a second dimension 406 (e.g., height), that is/are less in magnitude than a diameter dimension of the opening 204 of the receptacle 200. A third dimension 408 (e.g., length) of the body 418 package 400 may be greater in magnitude than the first and/or second dimension 404, 406. The package 400, which may be a sachet, may be in the form of a tube or “stick pack.”

When the packages 400 of salt are co-packaged with the device 100, the kit may comprise more than one of such packages 400. The number of packages 400 contained in the kit may be from one to one hundred, preferably from two to fifty, more preferably from three to thirty. Two or more packages 400, for example five packages 400, may be connected to each other. For example, one package 400 may be connected to another, for example at closed ends 410, 412 of the package 400, thereby making a string of a plurality of packages 400. Connecting the packages 400 may facilitate convenient packing for the manufacturer. When two or more packages 400 are connected, the packages 400 may be conveniently separable by a user. Such separation may be facilitated by a separation feature, such as a perforation or other line of weakness.

The package 400 may comprise indicia 414 on an outer surface 416. The indicia 414 may be printed on the outer surface 416 with any suitable ink. The ink may be of any suitable color and/or may be metallic. The indicia 414 may comprise branding/marketing indicia, instructional indicia, and/or safety indicia. The indicia 414 on the package 400 may correspond or even be partially the same as indicia 212 on the device 100. For example, it may be desirable for both the device 100 and the package 400 of salt to each include substantially the same or complementary brand name, logos, and/or color schemes. Thus, the indicia 212 of the nasal irrigation device 100 and the indicia 414 of the package 400 comprise at least one common element, such as branding/marketing indicia (e.g., brand names, logos, and/or color schemes).

### Processes

The present disclosure further relates to nasal irrigation processes. For example, the present disclosure relates to a process of using the nasal irrigation devices 100 according to the present disclosure. The process comprises the steps of: providing a salt solution to the interior volume 210 of the receptacle 200, preferably wherein the salt solution is prepared by providing a salt and water to the interior volume 210 and mixing; attaching the top 300 to the receptacle 200; inserting the tip 306 of the nozzle 302 into a human nostril; and dispensing at least a portion of the salt solution through the nozzle 302 to the human nostril. The human nostril preferably comprises nasosinal cavities that are in need of treatment, for example due to irritation, dryness, and/or congestion.

The salt may comprise a compound selected from the group consisting of sodium chloride, magnesium chloride, potassium chloride, calcium chloride, sodium bicarbonate, and a combination thereof. The salt preferably comprises sodium chloride. Thus, the salt solution may be a solution of sodium chloride, magnesium chloride, potassium chloride, calcium chloride, sodium bicarbonate, or a combination thereof. The salt solution preferably comprises a sodium chloride solution.

The salt may be dispensed directly into the receptacle 200, for example from a package 400 comprising salt. Water used to make the salt solution may be added before, during, or after the salt is provided to the receptacle 200. The water is preferably purified, distilled, or previously boiled water.

The present disclosure further relates to a process of treating a nasosinal cavity in need of treatment. The process includes the steps of providing a nasal irrigation device 100 as described herein, and dispensing a treatment solution, preferably a salt solution, from the device 100 to the nasosinal cavity in need of treatment.

The present disclosure further relates to manufacturing and/or assembly processes. For example, the present disclosure relates to a process of manufacturing a top 300 according to the present disclosure. The process may comprise the step of forming a top 300 according to the present disclosure, for example by injection molding using a thermoplastic material. The process of making a top 300 according to the present disclosure may comprise the steps of making a nozzle 302 and an intermediate part 304 as separate parts and then connecting them to form a top 300.

The present disclosure also relates to a process of making a nasal irrigation device 100 according to the present disclosure. The process may comprise the steps of providing a receptacle 200 according to the present disclosure and providing a top 300 according to the present disclosure. The process may comprise connecting the receptacle 200 and the device 300 to form a nasal irrigation device 100 according to the present disclosure. The process may comprise co-packaging the receptacle 200 and the top 300, which may optionally be connected. The process may further comprise co-packaging the receptacle 200 and the top 300 with at least one package of salt.

### COMBINATIONS

Specifically contemplated combinations of the disclosure are herein described in the following lettered paragraphs. These combinations are intended to be illustrative in nature and are not intended to be limiting.

A. A nasal irrigation device comprising: a receptacle, the receptacle comprising a body and an opening, the body comprising a base wall configured to enable the receptacle to stand upright on a flat surface, the body further comprising at least one side wall extending from the base wall and about a longitudinal axis that is substantially orthogonal to the base wall, wherein the base wall and the at least one side wall cooperate to define an interior volume of the body that is accessible through the opening; and a top that is selectively connectable to the receptacle in a manner that covers the opening when the top is attached to the receptacle, the top comprising a nozzle that is selectively rotatable relative to the receptacle, the nozzle comprising a dispensing orifice located at a tip of the nozzle, the nozzle further comprising a finger placement area and a projection, wherein the finger placement area comprises a centroid, wherein the projection is circumferentially located on the nozzle between 100° and 260°, preferably between 135° and 225°, preferably about 180°, from the centroid of the finger placement area, and wherein the finger placement area and the projection are configured to facilitate rotation of the nozzle relative to the receptacle.

B. A nasal irrigation device comprising: a receptacle, the receptacle comprising a body and an opening, the body comprising a base wall configured to enable the receptacle to stand upright on a flat surface, the body further comprising at least one side wall extending from the base wall and about a longitudinal axis that is substantially orthogonal to the base wall, wherein the base wall and the at least one side wall cooperate to define an interior volume of the body that is accessible through the opening; and a top that is selectively attachable to the receptacle in a manner that covers the opening when the top is attached to the receptacle, the top comprising a nozzle that is rotatable relative to the receptacle, the nozzle comprising a dispensing orifice located at a tip of the nozzle, the nozzle further comprising a finger placement area, wherein the tip of the nozzle tilts away from the longitudinal axis and towards the finger placement area.

C. A nasal irrigation device comprising: a receptacle, the receptacle comprising a body and an opening, the body comprising a base wall configured to enable the receptacle to stand upright on a flat surface, the body further comprising at least one side wall extending from the base wall and about a longitudinal axis that is substantially orthogonal to the base wall, wherein the base wall and the at least one side wall cooperate to define an interior volume of the body that is accessible through the opening; and a top that is selectively attachable to the receptacle in a manner that covers the opening when the top is attached to the receptacle, the top comprising a nozzle and an intermediate part located between the nozzle and the receptacle, wherein the nozzle is rotatable relative to the intermediate part, wherein the nozzle is characterized by a first color and the intermediate part is characterized by a second color that is different from the first color.

D. The nasal irrigation device according to any of paragraphs A-C, wherein the projection is circumferentially located on the nozzle between 135° and 225° from the centroid of the finger placement area.

E. The nasal irrigation device according to any of paragraphs A-D, wherein the projection is circumferentially located on the nozzle about 180° from the centroid of the finger placement area.

F. The nasal irrigation device according to any of paragraphs A-E, wherein the finger placement area comprises an outer edge, preferably a continuous outer edge, that defines at least a portion of a perimeter of the finger placement area.

G. The nasal irrigation device according to any of paragraphs A-F, wherein the finger placement area comprises a surface, wherein the surface comprises surface irregularities, preferably surface irregularities selected from the group consisting of ridges, bumps, depressions, and combinations thereof.

H. The nasal irrigation device according to any of paragraphs A-G, wherein the finger placement area comprises a surface, wherein the surface is partially or completely smooth.

I. The nasal irrigation device according to any preceding claim, wherein the finger placement area is characterized by at least one of the following characteristics: (a) being substantially concave in a longitudinal direction; (b) being substantially convex in a circumferential direction; (c) both (a) and (b).

J. The nasal irrigation device according to any of paragraphs A-I, wherein the top further comprises an intermediate part located between the receptacle and the nozzle, wherein the intermediate part is selectively connectable to the receptacle, and wherein the nozzle is selectively rotatable relative to the intermediate part.

K. The nasal irrigation device according to any of paragraphs A-J, wherein the intermediate part comprises an exterior surface, wherein the exterior surface comprises surface irregularities.

L. The nasal irrigation device according to any of paragraphs A-K, wherein the nozzle is characterized by a first color and the intermediate part is characterized by a second color that is different from the first color.

M. The nasal irrigation device according to any of paragraphs A-L, wherein the projection is in the form of a ridge, preferably a ridge that is longitudinally oriented.

N. The nasal irrigation device according to any of paragraphs A-M, wherein the projection is characterized by a projection length, a projection width, and a projection height.

O. The nasal irrigation device according to any of paragraphs A-N, wherein the device further comprises a tube that is selectively connectable to the top and that, when so connected, extends into the interior volume of the receptacle when the top is attached to the receptacle.

P. The nasal irrigation device according to any of paragraphs A-O, wherein the top comprises a flow rate adjustment means.

Q. The nasal irrigation device according to any of paragraphs A-P, wherein the nozzle comprises indicia, preferably wherein the indicia comprises instructional indicia, branding/marketing indicia, safety indicia, or combinations thereof, more preferably wherein the indicia comprises instructional indicia that comprises directional indicia, flow indicia, status indicia, or combinations thereof.

R. The nasal irrigation device according to any of paragraphs A-Q, wherein the tip of the nozzle tilts away from the longitudinal axis and towards the finger placement area.

S. A top for a nasal irrigation device, the top comprising a nozzle connected to an intermediate part, wherein the nozzle is selectively rotatable relative to the intermediate part; the nozzle comprising a dispensing orifice located at a tip of the nozzle, the nozzle further comprising a finger placement area and a projection, wherein the finger placement area comprises a centroid, wherein the projection is circumferentially located on the nozzle between 100° and 260°, preferably between 135° and 225°, preferably about 180°, from the centroid of the finger placement area, and wherein the finger placement area and the projection are configured to facilitate rotation of the nozzle relative to the intermediate part; the intermediate part being configured to be selectively connectable to a receptacle of a nasal irrigation device.

T. A nasal irrigation kit comprising: the nasal irrigation device according to any of paragraphs A-R; and a package comprising a salt, preferably wherein the salt comprises a compound selected from the group consisting of sodium chloride, magnesium chloride, potassium chloride, calcium chloride, sodium bicarbonate, and a combination thereof, preferably sodium chloride.

U. The nasal irrigation kit according to paragraph T, wherein the nasal irrigation device comprises indicia, wherein the package comprises indicia on a surface, and wherein the indicia of the nasal irrigation device and the indicia of the package comprise at least one common element.

V. A process of using the nasal irrigation device according to any of paragraphs A-R, wherein the process comprises the steps of: providing a salt solution to the interior volume of the receptacle of the nasal irrigation device, preferably wherein the salt solution is prepared by providing a salt and water to the interior volume of the receptacle and mixing; attaching the top to the receptacle; inserting the tip of the nozzle into a human nostril; and dispensing at least a portion of the salt solution through the nozzle to the human nostril.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be 10 made without departing from the spirit and scope of the invention. It is therefore intended to cover in the appended claims all such changes and modifications that are within the scope of this invention.

What is claimed is:

1. A nasal irrigation device comprising:
a receptacle, the receptacle comprising a body and an opening,
the body comprising a base wall configured to enable the receptacle to stand upright on a flat surface,
the body further comprising at least one side wall extending from the base wall and about a longitudinal axis that is substantially orthogonal to the base wall,
wherein the base wall and the at least one side wall cooperate to define an interior volume of the body that is accessible through the opening; and
a top that is selectively connectable to the receptacle in a manner that covers the opening when the top is attached to the receptacle,
the top comprising a nozzle that is selectively rotatable relative to the receptacle,
the nozzle comprising a dispensing orifice located at a tip of the nozzle,
the nozzle further comprising a finger placement area and a projection,
wherein the finger placement area comprises a centroid,
wherein the projection is circumferentially located on the nozzle between 100° and 260° from the centroid of the finger placement area, and
wherein the finger placement area and the projection are configured to facilitate rotation of the nozzle relative to the receptacle.

2. The nasal irrigation device according to claim 1, wherein the projection is circumferentially located on the nozzle between 135° and 225° from the centroid of the finger placement area.

3. The nasal irrigation device according to claim 1, wherein the projection is circumferentially located on the nozzle about 180° from the centroid of the finger placement area.

4. The nasal irrigation device according to claim 1, wherein the finger placement area comprises an outer edge that defines at least a portion of a perimeter of the finger placement area.

5. The nasal irrigation device according to claim 1, wherein the finger placement area comprises a surface, wherein the surface comprises surface irregularities.

6. The nasal irrigation device according to claim 1, wherein the finger placement area comprises a surface, wherein the surface is partially or completely smooth.

7. The nasal irrigation device according to claim 1, wherein the finger placement area is characterized by at least one of the following characteristics:
(a) being substantially concave in a longitudinal direction;
(b) being substantially convex in a circumferential direction;
(c) both (a) and (b).

8. The nasal irrigation device according to claim 1, wherein the top further comprises an intermediate part located between the receptacle and the nozzle,
wherein the intermediate part is selectively connectable to the receptacle, and
wherein the nozzle is selectively rotatable relative to the intermediate part.

9. The nasal irrigation device according to claim 1, wherein the intermediate part comprises an exterior surface,
wherein the exterior surface comprises surface irregularities.

10. The nasal irrigation device according to claim 8, wherein the nozzle is characterized by a first color and the intermediate part is characterized by a second color that is different from the first color.

11. The nasal irrigation device according to claim 1, wherein the projection is in the form of a ridge that is longitudinally oriented.

12. The nasal irrigation device according to claim 1, wherein the projection is characterized by a projection length, a projection width, and a projection height.

13. The nasal irrigation device according to claim 1, wherein the device further comprises a tube that is selectively connectable to the top and that, when so connected, extends into the interior volume of the receptacle when the top is attached to the receptacle.

14. The nasal irrigation device according to claim 1, wherein the top comprises a flow rate adjustment means.

15. The nasal irrigation device according to claim 1, wherein the nozzle comprises indicia,
wherein the indicia comprises instructional indicia, branding/marketing indicia, safety indicia, or combinations thereof.

16. The nasal irrigation device according to claim 15, wherein the indicia comprises instructional indicia that comprises directional indicia, flow indicia, status indicia, or combinations thereof.

17. The nasal irrigation device according to claim 1, wherein the tip of the nozzle tilts away from the longitudinal axis and towards the finger placement area.

18. A nasal irrigation device comprising:
a receptacle, the receptacle comprising a body and an opening,
the body comprising a base wall configured to enable the receptacle to stand upright on a flat surface,
the body further comprising at least one side wall extending from the base wall and
about a longitudinal axis that is substantially orthogonal to the base wall,
wherein the base wall and the at least one side wall cooperate to define an interior volume of the body that is accessible through the opening; and
a top that is selectively attachable to the receptacle in a manner that covers the opening when the top is attached to the receptacle,
the top comprising a nozzle that is rotatable relative to the receptacle,
the nozzle comprising a dispensing orifice located at a tip of the nozzle,
the nozzle further comprising a finger placement area,
wherein the tip of the nozzle tilts away from the longitudinal axis and towards the finger placement area.

19. A nasal irrigation device comprising:
a receptacle, the receptacle comprising a body and an opening,
the body comprising a base wall configured to enable the receptacle to stand upright on a flat surface,
the body further comprising at least one side wall extending from the base wall and about a longitudinal axis that is substantially orthogonal to the base wall,
wherein the base wall and the at least one side wall cooperate to define an interior volume of the body that is accessible through the opening; and
a top that is selectively attachable to the receptacle in a manner that covers the opening when the top is attached to the receptacle,
the top comprising a nozzle and an intermediate part located between the nozzle and the receptacle,
wherein the nozzle is rotatable relative to the intermediate part,
wherein the nozzle is characterized by a first color and the intermediate part is characterized by a second color that is different from the first color.

20. A top for a nasal irrigation device, the top comprising a nozzle connected to an intermediate part, wherein the nozzle is selectively rotatable relative to the intermediate part;
the nozzle comprising a dispensing orifice located at a tip of the nozzle,
the nozzle further comprising a finger placement area and a projection,
wherein the finger placement area comprises a centroid,
wherein the projection is circumferentially located on the nozzle between 100° and 260° from the centroid of the finger placement area, and
wherein the finger placement area and the projection are configured to facilitate rotation of the nozzle relative to the intermediate part;
the intermediate part being configured to be selectively connectable to a receptacle of a nasal irrigation device.

21. A nasal irrigation kit comprising:
the nasal irrigation device according to claim 1, and
a package comprising a salt.

22. The nasal irrigation kit according to claim 21, wherein the salt comprises a compound selected from the group consisting of sodium chloride, magnesium chloride, potassium chloride, calcium chloride, sodium bicarbonate, and a combination thereof.

23. The nasal irrigation kit according to claim 22, wherein the salt is sodium chloride.

24. The nasal irrigation kit according to claim 21,
wherein the nasal irrigation device comprises indicia,
wherein the package comprises indicia on a surface, and
wherein the indicia of the nasal irrigation device and the indicia of the package comprise at least one common element.

25. A process of using the nasal irrigation device according to claim 1, wherein the process comprises the steps of:
providing a salt solution to the interior volume of the receptacle of the nasal irrigation device;
attaching the top to the receptacle;
inserting the tip of the nozzle into a human nostril; and
dispensing at least a portion of the salt solution through the nozzle to the human nostril.

26. The process according to claim 25, wherein the salt solution is prepared by providing a salt and water to the interior volume of the receptacle, and mixing.
